(19) United States
(12) Patent Application Publication
(10) Pub. No.: US 2016/0077043 A1
(43) Pub. Date: Mar. 17, 2016

(54) BIOCHIP AND DEVICE FOR MEASURING BIOCHIP

(71) Applicant: Samsung Electro-Mechanics Co., Ltd., Suwon-si (KR)

(72) Inventor: Sang Hyun YI, Suwon-si (KR)

(73) Assignee: Samsung Electro-Mechanics Co., Ltd., Suwon-si (KR)

(21) Appl. No.: 14/664,577

(22) Filed: Mar. 20, 2015

(30) Foreign Application Priority Data
Sep. 16, 2014 (KR) 10-2014-0122535

(51) Int. Cl. G01N 27/403; G01N 33/487

(52) U.S. Cl. CPC G01N 27/403; G01N 33/48785

(57) ABSTRACT

There is provided a biochip including: a first substrate having a first surface in which a plurality of grooves are provided to accommodate at least one type of culture medium therein, and including a first electrode which is connected to the plurality of grooves; and a second substrate having a first surface in which a plurality of biomaterial fixing parts are provided to attach at least one type of biomaterial thereto, and including a second electrode which is connected to the plurality of biomaterial fixing parts. The biochip can rapidly and precisely measure a reaction of the biomaterial.

### CROSS-REFERENCE TO RELATED APPLICATION

This application claims the priority and benefit of Korean Patent Application No. 10-2014-0122535 filed on Sep. 16, 2014, with the Korean Intellectual Property Office, the disclosure of which is incorporated herein by reference.

### BACKGROUND

The present disclosure relates to a biochip configured to measure the degree of biomaterial culturing.

A biochip is used to culture biomaterials or to measure reactions of such biomaterials with drugs.

The reaction measurement of the biomaterials is performed by a method of observing the biochip with the naked eye or by a method of scanning the biochip using a digital image processing device. The former method has difficulty in measuring a fine reaction of the biomaterials, while the latter method have difficulties in performing such measurements in real time as well as increased costs due to equipment required for the device.

Therefore, the development of a biochip capable of precisely measuring reactions of the biomaterials with drugs in real time has been demanded.

As related art, there is provided Patent Document 1.

### RELATED ART DOCUMENT

(Patent Document 1) KR2012-138082 A

### SUMMARY

An aspect of the present disclosure may provide a biochip configured to precisely and rapidly measure reactions of biomaterials, and a device for measuring the biochip.

According to an aspect of the present disclosure, a biochip may include an electrode unit configured to measure electrical resistance of a biomaterial.

According to another aspect of the present disclosure, a device for measuring a biochip may include an measuring unit configured to measure a reaction state of a biomaterial using electrical resistance characteristics of the biomaterial cultured in the biochip.

### BRIEF DESCRIPTION OF DRAWINGS

The above and other aspects, features and advantages of the present disclosure will be more clearly understood from the following detailed description taken in conjunction with the accompanying drawings, in which:

FIG. 1 is an exploded perspective view of a biochip according to an exemplary embodiment in the present disclosure;

FIG. 2 is a bottom perspective view of the biochip illustrated in FIG. 1;

FIG. 3 is an assembled perspective view of the biochip illustrated in FIG. 1;

FIG. 4 is a cross-sectional view of the biochip illustrated in FIG. 3, taken along line A-A;

FIG. 5 is an exploded perspective view of biochip according to another exemplary embodiment in the present disclosure;

FIG. 6 is a bottom perspective view of the biochip illustrated in FIG. 5;

FIG. 7 is an assembled perspective view of the biochip illustrated in FIG. 5;

FIG. 8 is a cross-sectional view of the biochip illustrated in FIG. 7, taken along line B-B;

FIG. 9 is an exploded perspective view of a biochip according to another exemplary embodiment in the present disclosure;

FIG. 10 is a bottom view of a first substrate illustrated in FIG. 9;

FIG. 11 is an assembled perspective view of the biochip illustrated in FIG. 9;

FIG. 12 is a cross-sectional view of the biochip illustrated in FIG. 11, taken along line C-C;

FIG. 13 is a cross-sectional view of the biochip illustrated in FIG. 11, taken along line D-D;

FIG. 14 is an exploded perspective view of a biochip according to another exemplary embodiment in the present disclosure;

FIG. 15 is a bottom view of a first substrate illustrated in FIG. 14;

FIG. 16 is an assembled perspective view of the biochip illustrated in FIG. 14;

FIG. 17 is a cross-sectional view of the biochip illustrated in FIG. 16, taken along line E-E;

FIG. 18 is a cross-sectional view of the biochip illustrated in FIG. 16, taken along line F-F; and

FIG. 19 is a configuration diagram a device for measuring a biochip according to an exemplary embodiment in the present disclosure.

### DETAILED DESCRIPTION

Exemplary embodiments of the present disclosure will now be described in detail with reference to the accompanying drawings.

The disclosure may, however, be embodied in many different forms and should not be construed as being limited to the embodiments set forth herein. Rather, these embodiments are provided so that this disclosure will be thorough and complete, and will fully convey the scope of the disclosure to those skilled in the art.

In the drawings, the shapes and dimensions of elements may be exaggerated for clarity, and the same reference numerals will be used throughout to designate the same or like elements.

Further, the term “biomaterial” as used in the present specification may include cells, proteins, DNA, RNA, and the like, of animals and plants, including human beings. Further, “biomaterial” may also refer to pathogens, pathogenic cells, and the like, generated from animals and plants.

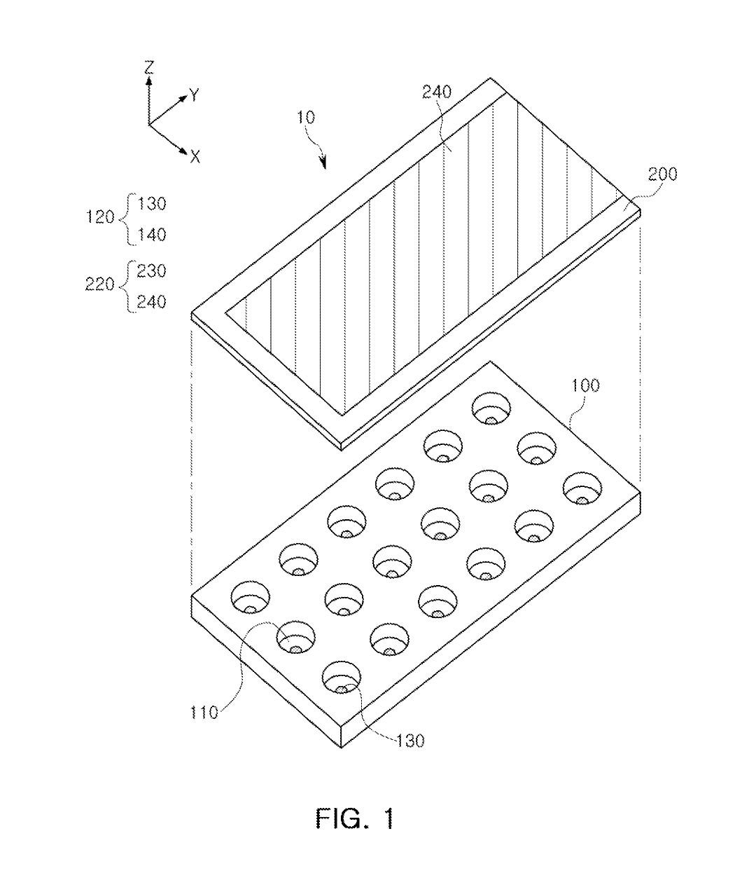

A biochip according to an exemplary embodiment will be described with reference to FIG. 1.

A biochip 10 may include a first substrate 100 and a second substrate 200. For example, the biochip 10 may include the first substrate 100 in which at least one type of culture medium is stored, and the second substrate 200 to which at least one type of biomaterial is attached. The biochip 10 may include a first electrode 120 and a second electrode 220. For example, the biochip 10 may include the first electrode 120 formed on the first substrate 100, and the second electrode 220 formed on the second substrate 200.

The first substrate 100 may be formed in a thin plate form. For example, the first substrate 100 may be formed in a rectangular form having a predetermined thickness. The first substrate 100 may be formed of a material having excellent chemical resistance and corrosion resistance. For example, the first substrate 100 may be formed of a material such as plastic, glass, silicon, or the like.

The first substrate 100 may have a plurality of grooves 110 formed therein. For example, the plurality of grooves 110 for accommodating the culture medium may be formed in a first surface of the first substrate 100. The first substrate 100 may be coated with a plurality of materials. For example, the grooves 110 may be coated with a hydrophilic material, and portions other than the grooves 110 may be coated with a hydrophobic material.

The first substrate 100 may have the first electrode 120 formed thereon. For example, the groove 110 of the first substrate 100 may have a first internal electrode 130 formed therein, wherein the first internal electrode 130 is a part of the first electrode 120. The first internal electrode 130 may be extended from the groove 110 to a second surface of the first substrate 100.

The second substrate 200 may be formed in a thin plate form, similar to the first substrate 100. For example, the second substrate 200 may be formed in a rectangular form having a very thin thickness. The second substrate 200 may be formed of a material having excellent chemical resistance and corrosion resistance. For example, the second substrate 200 may be formed of a material such as plastic, glass, silicon, or the like.

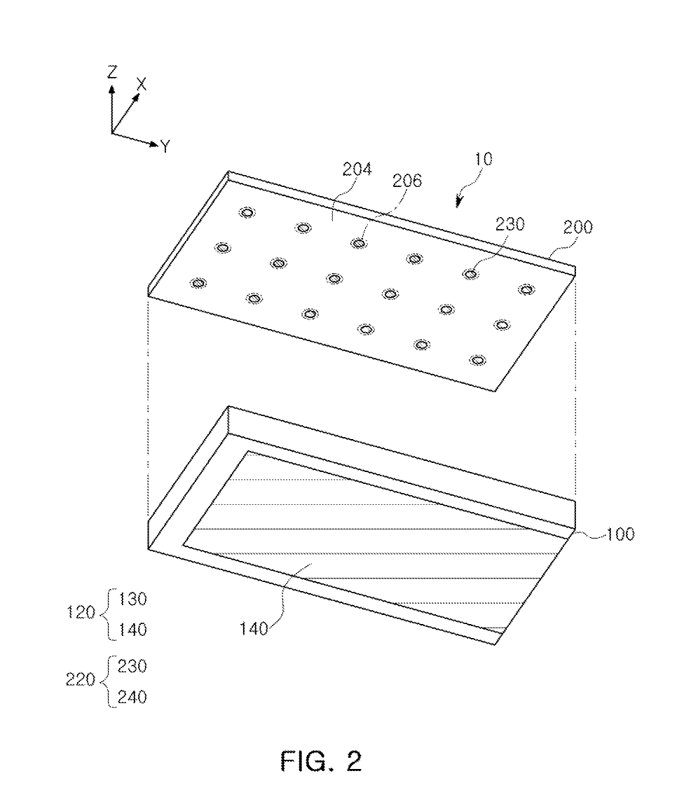

The second substrate 200 may be coated with a plurality of materials. For example, a portion of the second substrate 200 may be coated with a hydrophobic material, and other portions may be coated with a hydrophilic material (a description thereof will be provided below with reference to FIG. 2).

The second substrate 200 may have the second electrode 220 formed thereon. For example, the second substrate 200 may have a second external electrode 240 formed on the first surface thereof, wherein the second external electrode 240 is a part of the second electrode 220. The second external electrode 240 may be formed on the first surface of the second substrate 200 to be wide and may be extended in a length direction (a direction of a Y axis based on FIG. 1) of the second substrate 200 to be long.

A bottom shape of the biochip will be described with reference to FIG. 2.

The biochip 10 may have a plurality of electrodes 120 and 220 formed thereon. For example, the first substrate 100 may have the first electrode 120 formed thereon and the second substrate 200 may have the second electrode 220 formed thereon.

The first electrode 120 may be formed in the groove 110 of the first substrate 100 and on the second surface of the first substrate 100. For example, the first internal electrode 130 of the first electrode 120 may be formed in each of the grooves 110 as described above, and the first external electrode 140 of the first electrode 120 may be formed on the second surface of the first substrate 100. The first external electrode 140 may be formed to be connected to a plurality of first internal electrodes 130. For example, the first external electrode 140 may be formed to be wide to be connected all of the first internal electrodes 130 that are extended from the groove 110 to the first surface of the first substrate 100.

The second electrode 220 may be each formed on the first surface and the second surface of the second substrate 200. For example, the second internal electrode 230 of the second electrode 220 may be formed to be long from the second surface of the second substrate 200 to the first surface thereof, and the second external electrode 240 of the second electrode 220 may be formed on the first surface of the second substrate 200 to be wide. The second external electrode 240 may be formed to be connected to a plurality of second internal electrodes 230. For example, the second external electrode 240 may be formed to be wide to be connected all of the second internal electrodes 230 that are extended to the first surface of the second substrate 200.

The second surface of the second substrate 200 may be partitioned into a plurality of regions. For example, the second surface of the second substrate 200 may be partitioned into a region to which the biomaterial is attached (for reference, corresponding to a biomaterial fixing part described in the claims) and other regions. Here, the latter may be a first region 204 which is coated with the hydrophobic material, and the former is a second region 206 which is coated with the hydrophilic material.

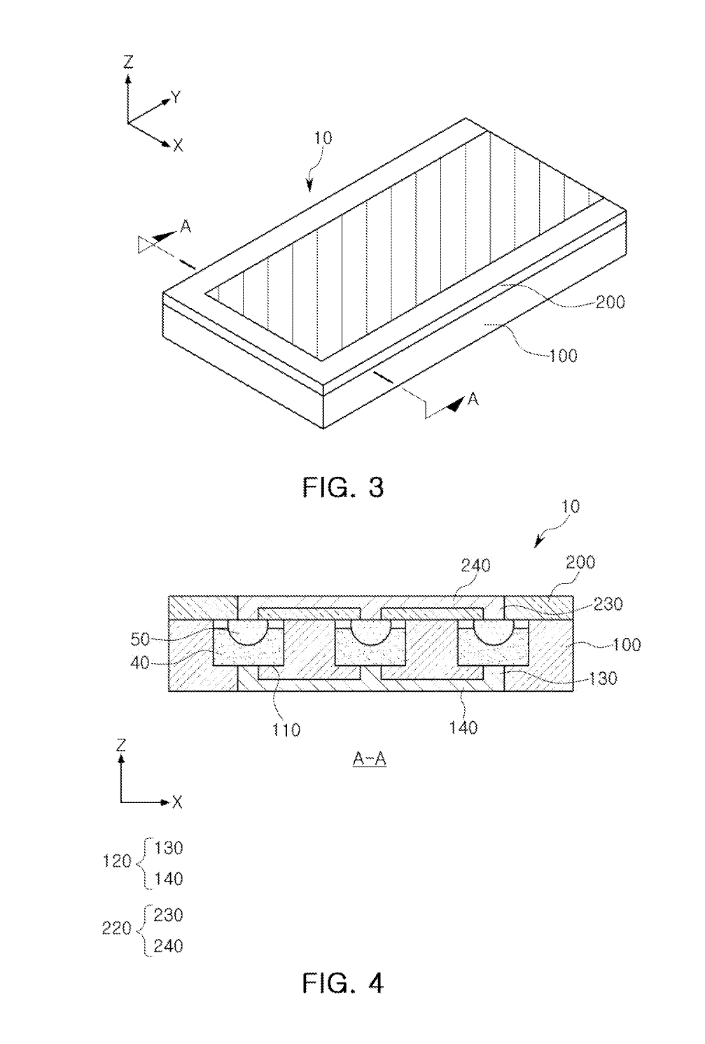

An assembled shape of the biochip will be described with reference to FIG. 3.

The biochip 10 may be formed in a shape in which the first substrate 100 and the second substrate 200 are assembled with each other. For example, the biochip 10 may be formed by the first surface of the first substrate 100 and the second surface of the second substrate 200 that are assembled with each other to be in contact with each other.

The biochip 10 formed as described above may be used for the culture of the biomaterials or a drug reaction experiment of the biomaterials.

A shape of a cross section of the biochip taken along line A-A will be described with reference to FIG. 4.

The biochip 10 may be formed in the assembled form of the first substrate 100 and the second substrate 200 as described above.

The first substrate 100 may be disposed below the biochip 10 and may accommodate a culture medium or a drug 40. For example, the groove 110 of the first substrate 100 may accommodate the culture medium or the drug 40. The first substrate 100 may include the first electrode 120 that transmits or senses an electrical signal which is necessary for the experiment of the biomaterials. For example, the first internal electrodes 130 that are extended in one direction (a direction of a Z axis based on FIG. 4) to be long may be each formed in the grooves 110 of the first substrate 100, and one first external electrode 140 that is connected to the plurality of first internal electrodes 130 may be formed on a lower surface of the first substrate 100.

The second substrate 200 may be disposed over the biochip 10 and may accommodate a biomaterial 50. For example, the biomaterial 50 may be attached to the second substrate 200. For reference, the attachment of the biomaterial 50 may be performed by a separate fixing material. The second substrate 200 may include the second electrode 220 that transmits or senses an electrical signal which is necessary for the experiment of the biomaterials. For example, the second internal electrodes 230 that are extended in one direction (a direction of a Z axis based on FIG. 4) from a region to which the biomaterial 50 is attached to be long may be each formed on the second surface of the second substrate 200, and one second external electrode 240 that is connected to the plurality of second internal electrodes 230 may be formed on an upper surface of the second substrate 200.

The biochip 10 configured as described above may measure electrical characteristics (e.g., electrical resistance, impedance, and the like) of the biomaterial 50 through the first electrode 120 and the second electrode 220. Further, the biochip 10 may measure a culture state or a drug reaction state of the biomaterial 50 through the measured electrical characteristics values.

Hereinafter, another exemplary embodiment in the biochip will be described. For reference, in the description of another exemplary embodiment in the bio chip, the same components as those of an exemplary embodiment described above will be denoted by the same reference numerals as those of an exemplary embodiment described above and a description thereof will be omitted.

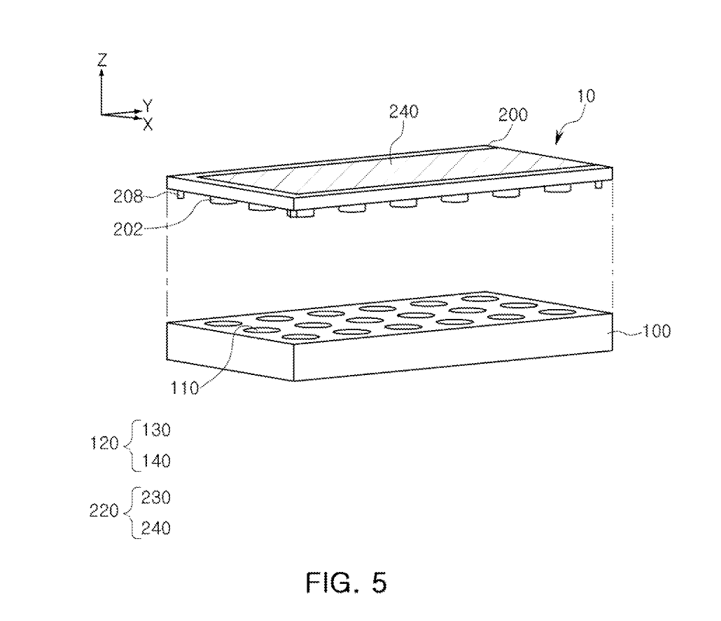

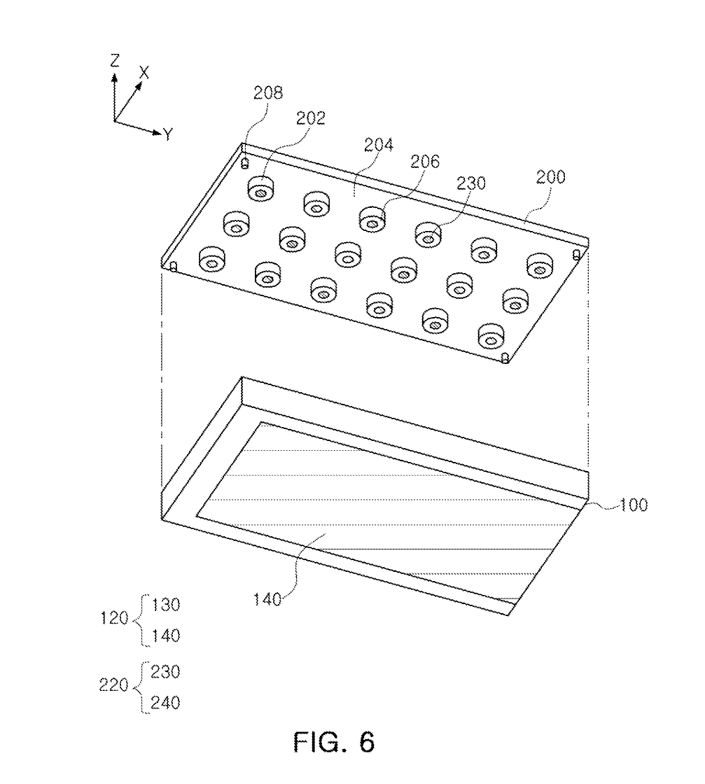

A biochip according to another exemplary embodiment will be described with reference to FIGS. 5 and 6.

The biochip 10 according to the present exemplary embodiment may be distinguished from an exemplary embodiment described above in a shape of the second substrate 200.

The second substrate 200 may have a plurality of protrusions 202 formed thereon. For example, the plurality of protrusions 202 may be formed on the second surface of the second substrate 200. The second internal electrode 230 may be formed in each of the protrusions 202 (see FIG. 6). The protrusion 202 formed as described above may provide the second region 206 to which the biomaterial is attached.

The second substrate 200 may have two or more interval maintaining members 208 formed thereon. For example, four interval maintaining members 208 may be formed on the second surface of the second substrate 200. The interval maintaining members 208 formed as described above may maintain an interval between the first surface of the first substrate 100 and the second surface of the second substrate 200 to be constant.

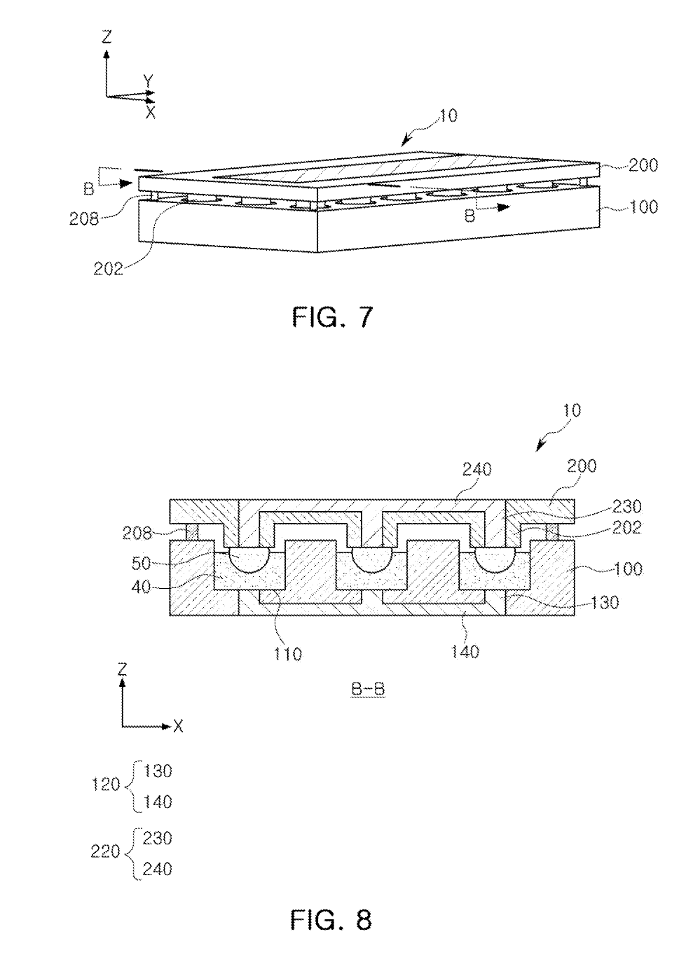

An assemble shape and a cross-section shape of the biochip will be described with reference to FIGS. 7 and 8.

The biochip 10 may be formed by the assembly of the first substrate 100 and the second substrate 200. For example, the biochip 10 may have a configuration in which the protrusion 202 of the second substrate 200 is assembled with the groove 110 of the first substrate 100 to substantially face each other. Here, the protrusion 202 may be partially inserted into the groove 110. However, the protrusion 202 is not necessarily inserted into the groove 110. For example, an end portion of the protrusion 202 may also be positioned to be higher than the upper surface of the groove 110.

The first substrate 100 and the second substrate 200 may be partially in contact with each other by the interval maintaining member 208. For example, the first substrate 100 and the second substrate 200 may be assembled with each other so as not in contact with any portion except for the interval maintaining member 208. Since the above-mentioned assembled structure significantly reduces a friction area between the first substrate 100 and the second substrate 200, it may easily perform an assembly and a separation between the first substrate 100 and the second substrate 200.

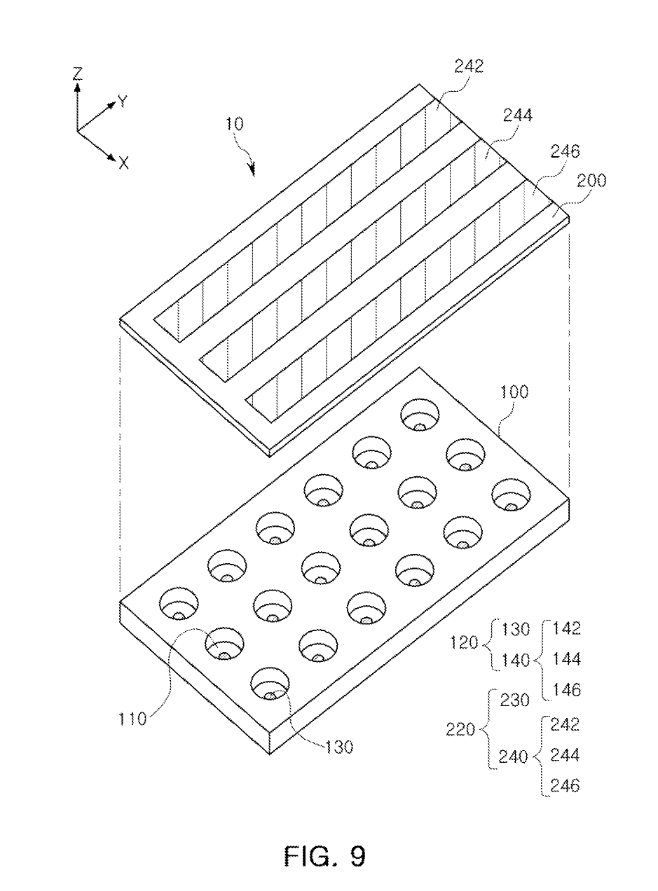

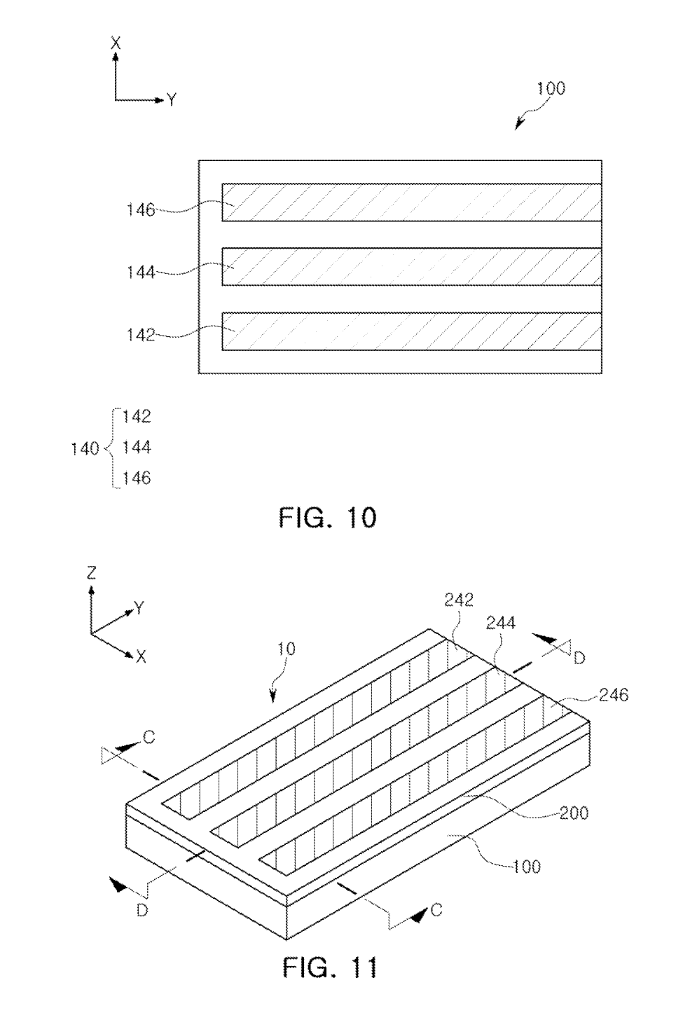

A biochip according to another exemplary embodiment will be described with reference to FIGS. 9 and 10.

The biochip 10 according to the present exemplary embodiment may be distinguished from an exemplary embodiment described above in formed shapes of the electrodes 120 and 220.

The first electrode 120 may include a plurality of first internal electrodes 130 and a plurality of first external electrodes 140 (142, 144, and 146). For example, the plurality of first internal electrodes 130 may be formed in each of the grooves 110, and the plurality of first external electrodes 140 (142, 144, and 146) may be formed on the second surface of the first substrate 100 (see FIG. 10). The first internal electrode 130 may be formed along a thickness direction (a direction of a Z axis based on FIG. 9) of the first substrate 100. For example, the first internal electrode 130 may be formed from a bottom surface of the groove 110 to the second surface of the first substrate 100 to be long. The first external electrodes 140 (142, 144, and 146) may be formed along a length direction (a direction of a Y axis based on FIG. 9) of the first substrate 100 to be long. For example, the respective first external electrodes 140 (142, 144, and 146) may be formed to be parallel to each other along the direction of the Y axis to be able to be connected the first internal electrodes 130 having the same number as that of the first external electrodes.

The second electrode 220 may include a plurality of second internal electrodes 230 and a plurality of second external electrodes 240 (242, 244, and 246). For example, the plurality of second internal electrodes 230 may be formed on the second surface of the second substrate, and the plurality of second external electrodes 240 (242, 244, and 246) may be formed on the first surface of the second substrate 200. The second internal electrode 230 may be formed along a thickness direction (a direction of a Z axis based on FIG. 9) of the second substrate 200. For example, the second internal electrode 230 may be formed from the second surface of the second substrate to the first surface of the second substrate 200 to be long. The second external electrodes 240 (242, 244, and 246) may be formed along a length direction (a direction of a Y axis based on FIG. 9) of the second substrate 200 to be long. For example, the respective second external electrodes 240 (242, 244, and 246) may be formed to be parallel to each other along the direction of the Y axis to be able to be connected the second internal electrodes 230 having the same number as that of the second external electrodes.

The biochip configured as described above may have a structure in which the plurality of internal electrodes 130 and 230 are partitioned by three external electrodes 140 and 240.

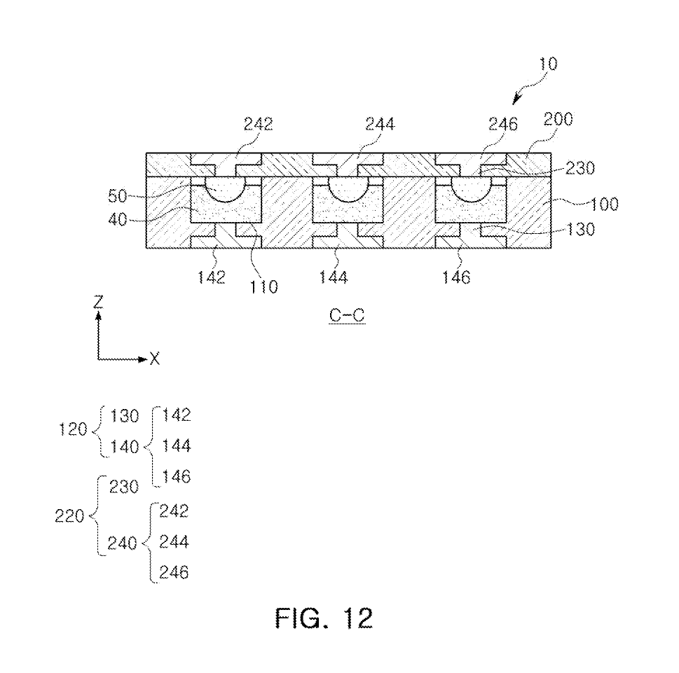

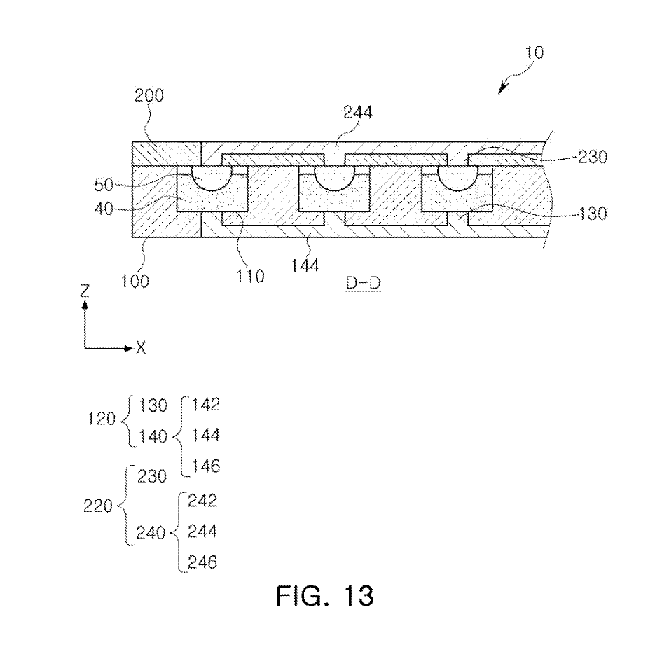

An assemble shape and a cross-section shape of the biochip will be described with reference to FIG. 11 through 13.

The biochip 10 may be formed by assembling the first substrate 100 having the plurality of first external electrodes 140 (142, 144, and 146) and the second substrate 200 having the plurality of second external electrodes 240 (242, 244, and 246). For example, the biochip 10 may be formed by assembling the first substrate 100 having three first external electrodes 140 (142, 144, and 146) and the second substrate 200 having the second external electrodes 240 (242, 244, and 246) having the same number as the first external electrodes. The first external electrodes 140 (142, 144, and 146) and the second external electrodes 240 (242, 244, and 246) may be formed to be in parallel to each other. For example, the first external electrodes 140 (142, 144, and 146) may be formed along a length direction (a direction of a Y axis based on FIG. 11) of the first substrate 100 to be long, and the second external electrodes 240 (242, 244, and 246) may be formed along a length direction (a direction of a Y axis based on FIG. 11) of the second substrate 200 to be long.

The biochip 10 formed as described above may be used to simultaneously experiment a plurality of biomaterials or drugs. For example, the biochip 10 may measure a first type of biomaterial 50 and drug 40 using the first external electrode 142 and the second external electrode 242, may measure a second type of biomaterial 50 and drug 40 using the first external electrode 144 and the second external electrode 244, and may measure a third type of biomaterial 50 and drug 40 using the first external electrode 146 and the second external electrode 246.

Therefore, an effort involved in separately performing experiments for various biomaterials and various drug reactions may be saved.

Meanwhile, although the present exemplary embodiment describes a case in which the plurality of external electrodes 140 and 240 are extended along the length direction (the direction of Y axis based on FIG. 11) of the substrates 100 and 200, the plurality of external electrodes 140 and 240 may be extended along a width direction (a direction of an X axis based on FIG. 11) of the substrates 100 and 200, as needed. In this case, experiments for more various biomaterials and drug reactions may be performed using the biochip 10.

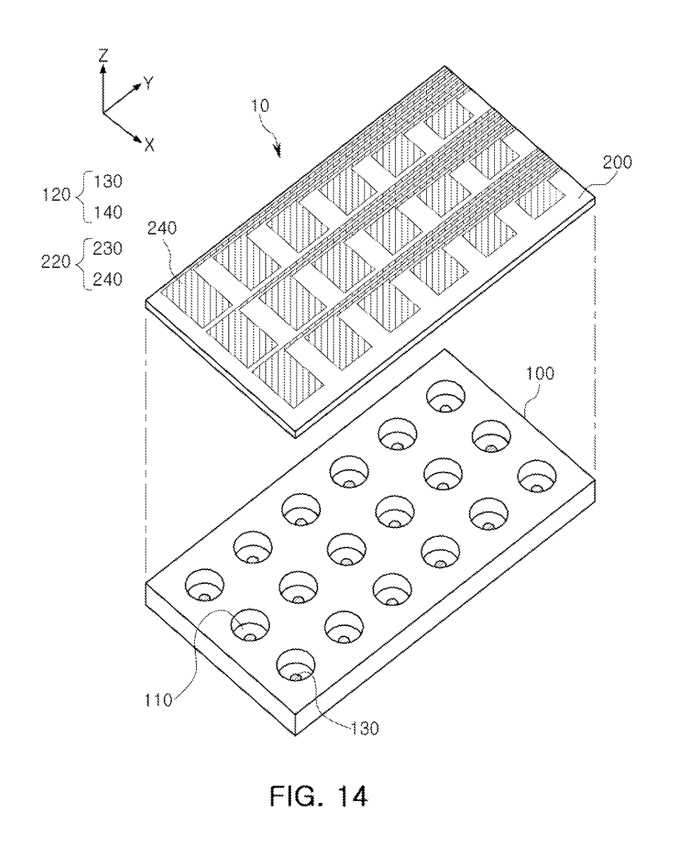

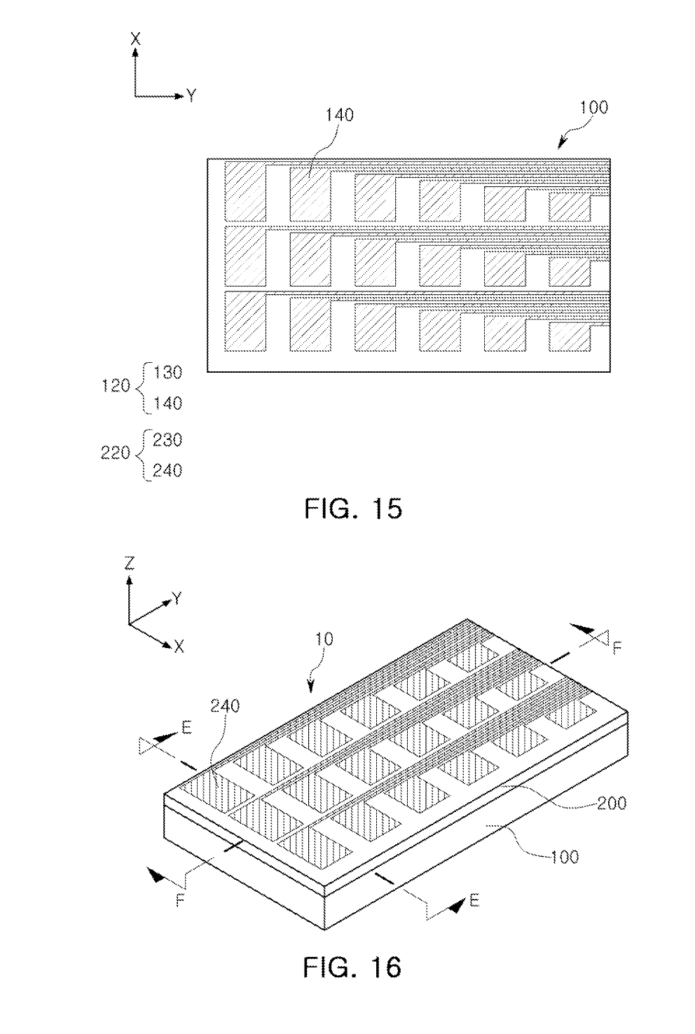

A biochip according to another exemplary embodiment will be described with reference to FIGS. 14 and 15.

The biochip 10 according to the present exemplary embodiment may be distinguished from an exemplary embodiment described above in formed shapes of the external electrodes 140 and 240. For example, the first external electrode 140 and the second external electrode 240 may be formed to correspond to the grooves 110 of the first substrate 100 as illustrated in FIGS. 14 and 15. Further, the first external electrode 140 and the second external electrode 240 may be formed to have the same number as that of grooves 110 of the first substrate 100 as illustrated in FIGS. 14 and 15.

An assemble shape and a cross-section shape of the biochip will be described with reference to FIG. 16 through 18.

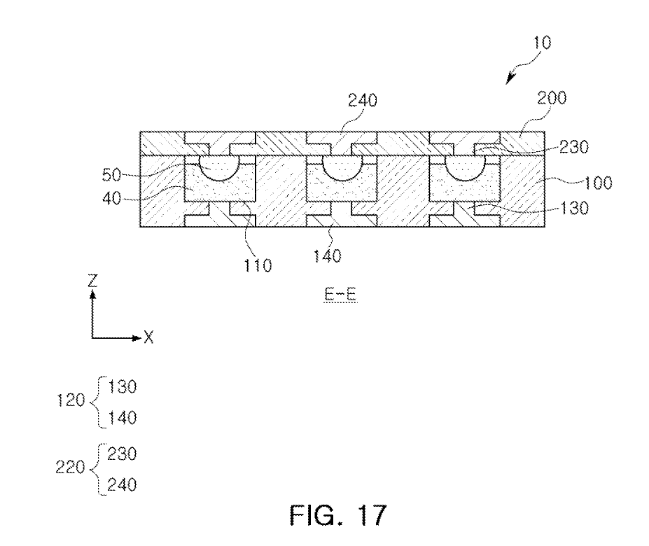

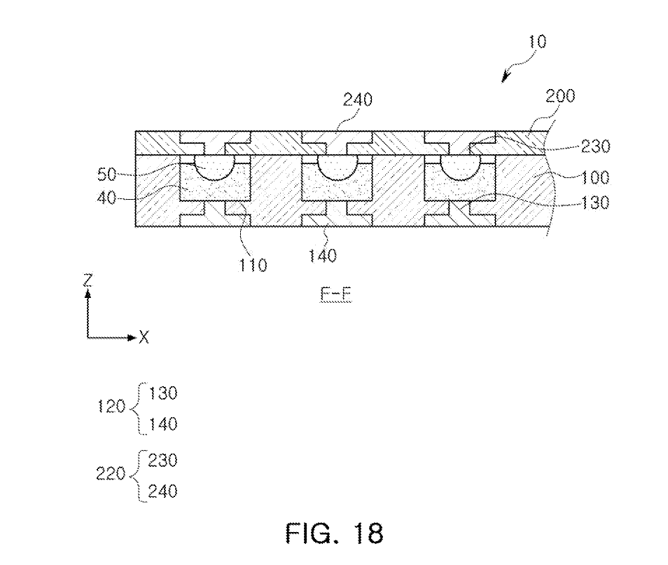

The biochip 10 may be configured to separately measure the biomaterials cultured in the plurality of grooves 110. For example, the first electrode 120 and the second electrode 220 are each separated from each other with respect to the length direction (the direction of the Y axis) and the width direction (the direction of the X axis) of the substrates 100 and 200 (see FIGS. 17 and 18).

Therefore, the biochip 10 according to the present exemplary embodiment may simultaneously measure the reaction experiments of the biomaterials for different culture mediums or drugs by attaching the same type of biomaterial onto the second substrate 200 and storing different types of culture mediums or drugs in the grooves 110 of the first substrate 100. Further, the biochip 10 according to the present exemplary embodiment may simultaneously measure the reaction experiments of various biomaterials for the same type of culture medium or drug by attaching different types of biomaterials onto the second substrate 200 and storing the same type of culture medium or drug in the grooves 110 of the first substrate 100.

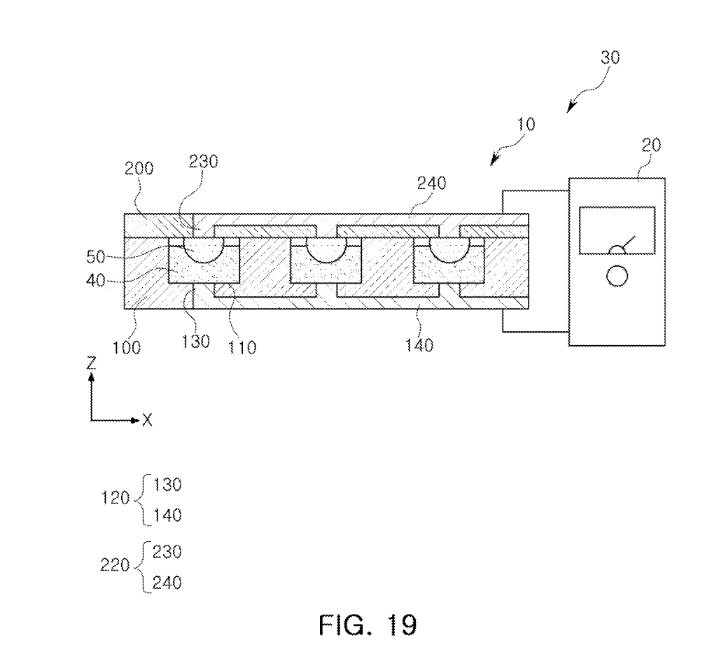

FIG. 19 is a configuration diagram a device for measuring a biochip according to an exemplary embodiment in the present disclosure.

A device 30 for measuring a biochip may include one of the bio chips 10 according to various exemplary embodiments described above and a measuring unit 20.

The measuring unit 20 may be connected to the external electrodes 140 and 240 of the substrates 100 and 200. The measuring unit 20 may measure a culture state of the biomaterial or a reaction state of the biomaterial and the drug. For example, the measuring unit 20 may measure electrical characteristics of the biomaterial and the culture medium using the external electrodes 140 and 240, and consequently, may measure the culture state of the biomaterial or the reaction state of the biomaterial and the drug. To this end, the measuring unit 20 may include a memory element in which basis information on the biomaterial, the culture medium, the drug, and the like of an experiment target is stored. Further, the measuring unit 20 may include a computing element capable of determining the culture state of the biomaterial or the reaction state of the biomaterial and the drug by comparing the basic information with measured information.

The device for measuring the biochip configured as described above may rapidly and precisely measure a state of the cultured biomaterial using the biochip.

Hereinafter, a principle and a method for measuring the biomaterial using the device for measuring the biochip will be described.

The device 30 for measuring the biochip may measure the state of the biomaterial using impedance of the biomaterial.

For example, in the case in which the first internal electrode 120 and the second internal electrode 220 are supplied with an alternating current, since cells configuring the biomaterial are charged with charges, the biomaterial may have impedance. Then, the device 30 for measuring the biochip may indirectly determine the state of the biomaterial by measuring the impedance of the biomaterial. For example, since the cell having an intact cell membrane and a membrane potential acts as a capacitor to accumulate the charges in the cell, it may have a high impedance value, but since the cell having a cell membrane which is not intact and having a degraded function of mitochondria does not smoothly generate energy for maintaining a cell membrane potential, which causes a decrease in a capacitive phenomenon, it may have a low impedance value.

Therefore, in the case in which the impedance value of the biomaterial measured by the device 30 for measuring the biochip is high, it may be determined that the number of cells configuring the biomaterial is large, and in the case in which the impedance value of the biomaterial is low, it may be determined that the number of cells configuring the biomaterial is small. That is, the device 30 for measuring the biochip may directly or indirectly determine a state of a bio cell membrane, whether or not energy of the cell is generated, and the like, using the impedance difference described above.

Further, the device 30 for measuring the biochip may measure a cell state of the biomaterial by varying a frequency of a current. For example, the device 30 for measuring the biochip may determine the state of the biomaterial through a change in the frequency over time after applying a predetermined voltage or alternating current to the biomaterial.

As set forth above, according to exemplary embodiments of the present disclosure, the reaction of the biomaterials may be rapidly and precisely measured.

While exemplary embodiments have been shown and described above, it will be apparent to those skilled in the art that modifications and variations could be made without departing from the scope of the present invention as defined by the appended claims.

What is claimed is:

1. A biochip comprising:
a first substrate having a first surface in which a plurality of grooves are provided to accommodate at least one type of culture medium therein, and including a first electrode which is connected to the plurality of grooves; and
a second substrate having a first surface in which a plurality of biomaterial fixing parts are provided to attach at least one type of biomaterial thereto, and including a second electrode which is connected to the plurality of biomaterial fixing parts.

2. The biochip of claim 1, wherein the first electrode is elongated in a second surface of the first substrate in a length direction of the first substrate, and
the second electrode is elongated in a second surface of the second substrate in a length direction of the second substrate.

3. The biochip of claim 1, wherein the first electrode includes:
a plurality of first internal electrodes extended from the plurality of grooves to a second surface of the first substrate; and
a first external electrode disposed on the second surface of the first substrate and connected to the plurality of first internal electrodes.

4. The biochip of claim 1, wherein the first electrode includes:
a plurality of first internal electrodes extended from the plurality of grooves to a second surface of the first substrate; and
a plurality of first external electrodes disposed on the second surface of the first substrate and connected to at least one of the first internal electrodes.

5. The biochip of claim 1, wherein the second electrode includes:
a plurality of second internal electrodes extended from the plurality of biomaterial fixing parts to a second surface of the second substrate; and
a second external electrode disposed on the second surface of the second substrate and connected to the plurality of second internal electrodes.

6. The biochip of claim 1, wherein the second electrode includes:
a plurality of second internal electrodes extended from the plurality of biomaterial fixing parts to a second surface of the second substrate; and
a plurality of second external electrodes disposed on the second surface of the second substrate and connected to at least one of the second internal electrodes.

7. The biochip of claim 1, wherein the plurality of biomaterial fixing parts are disposed on a plurality of protrusions protruding from the first surface of the second substrate.

8. The biochip of claim 1, wherein the biomaterial fixing parts are regions coated with a hydrophilic material.

9. The biochip of claim 1, wherein the biomaterial fixing parts are regions surrounded by a hydrophobic material.

10. A device for measuring a biochip, the device comprising:
a first substrate having a first surface in which a plurality of grooves are provided to accommodate at least one type of culture medium therein, and including a first electrode which is connected to the plurality of grooves;
a second substrate having a first surface in which a plurality of biomaterial fixing parts are provided to attach at least one type of biomaterial thereto, and including a second electrode which is connected to the plurality of biomaterial fixing parts; and
a measuring unit configured to be connected to the first and second electrodes and to measure electrical resistances of the culture medium and the biomaterial disposed between the grooves and the biomaterial fixing parts.
